(19) United States
(12) Patent Application Publication
(10) Pub. No.: US 2018/0236174 A1
(43) Pub. Date: Aug. 23, 2018

(54) Nesting Plunger Tip with Seal Bypass for Syringe Dispensing

(71) Applicants: Mark Curtis Archambeault, Hamilton (CA); Aaron James Tufgar, Hamilton (CA)

(72) Inventors: Mark Curtis Archambeault, Hamilton (CA); Aaron James Tufgar, Hamilton (CA)

(21) Appl. No.: 15/438,811

(22) Filed: Feb. 22, 2017

(51) Int. Cl. A61M 5/315

(52) U.S. Cl. CPC A61M 5/31513; A61M 2005/31516

(57) ABSTRACT

The present disclosure provides a novel nesting plunger tip that can be installed onto a loose syringe plunger to create a syringe package that allows dispensing and abrupt stoppage of dispensing by creating negative pressure inside the syringe. This negative pressure can be achieved because the nesting plunger tip seats inside the syringe stopper and holds onto the stopper firmly when the plunger is pulled back, which creates negative pressure on the material in the syringe by pulling back on it.

The nesting plunger tip can also be left off the plunger and the plunger can then be taken out of the syringe to allow a pneumatic dispensing adapter to be attached to provide positive or negative air pressure behind the syringe stopper.

The nesting plunger tip allows a single syringe with plunger to be used for either manual hand dispensing or pneumatic dispensing.

### CROSS-REFERENCE TO RELATED APPLICATIONS

The present disclosure claims priority from U.S. provisional patent application No. 62/298,084 filed on Feb. 22, 2016, the contents of which are incorporated herein by reference.

### BACKGROUND OF THE INVENTION

### 1. Field of the Invention

This invention relates generally to the packaging and dispensing of solder paste and solder flux and more particularly to the packaging and dispensing of solder paste and solder flux in syringes.

### 2. Description of the Related Art

It is generally known in the art to use a syringe stopper attached to a plunger handle in order to allow precise dispensing from a syringe and to allow for pulling back to create negative pressure to abruptly stop the flow of material. For example U.S. Pat. Nos. 4,215,701, 4,986,820, 8,740,854.

Some inventions have been published that include two part plungers, but with different designs and different utility than the invention presently disclosed. For example U.S. Pat. No. 9,532,742.

### BRIEF SUMMARY OF THE INVENTION

The invention disclosed solves the issue of a syringe of solder paste or solder flux only being suitable for use in either manual dispensing or pneumatic dispensing. In manual dispensing, if the ability to abruptly stop the flow of solder paste or solder flux from the syringe is required, a captive plunger that comes permanently affixed to the syringe stopper is used. This allows the syringe to be manually hand dispensed and the flow of solder paste or solder flux to be abruptly stopped when needed, but makes the syringe unusable for pneumatic dispensing.

If a syringe is setup for pneumatic dispensing, then the plunger is not affixed to the syringe stopper and when trying to manually dispense from a syringe using a non-affixed plunger, it is not possible to create negative pressure by pulling back on the syringe plunger, so the flow of solder paste or solder flux cannot be abruptly stopped. However, the loose plunger does allow pneumatic dispensing since the loose plunger can be removed from the syringe and the syringe can then be mated with a pneumatic dispensing adapter to apply positive or negative air pressure.

The nesting plunger tip invention disclosed herein allows the end user of a syringe to make the determination of which way they want to use the syringe, either hand dispensing, if they install the nesting plunger tip, or by pneumatic dispensing, if they leave the nesting plunger tip and plunger out of the syringe. The main advantage is that the end user can decide which way to assemble and use the nesting plunger tip depending on whether they want to do manual hand dispensing or pneumatic dispensing. This allows the manufacturer of the syringe to assemble and pack only one type of syringe, instead of two kinds which are normally required in order to serve both the manual hand dispensing and the pneumatic dispensing markets.

### BRIEF DESCRIPTION OF THE DRAWINGS

FIG. 1 shows the embodiment of the nesting plunger tip 1 with a notch in the side 2 that creates a seal bypass which allows air to escape as the nesting plunger tip 1 is inserted into a syringe stopper. The nesting plunger tip 1 is first pushed onto the plunger which fits inside the backside cavity 3 of the nesting plunger tip 1.

FIG. 2 shows the embodiment of the nesting plunger tip 1 with half the tip cut away to show the internal cavity 3 in more detail.

FIG. 3 shows the embodiment of the nesting plunger tip 1 with syringe 4, syringe stopper 5, and syringe plunger 6 all displayed separately.

FIG. 4 shows the embodiment of the nesting plunger tip 1 installed onto the tip of the syringe plunger 6, as well as the syringe stopper 5 inserted into the syringe 4.

FIG. 5 shows the nesting plunger tip 1 installed onto the syringe plunger 6 and being inserted into the syringe 4 to nest with the syringe stopper 5.

FIG. 6 shows the nesting plunger tip 1 installed onto the syringe plunger 6 and fully nested into the syringe stopper 5. This then allows the syringe stopper 5 to be pushed in or pulled out by pushing in or pulling out the syringe plunger 6.

### DETAILED DESCRIPTION OF THE INVENTION

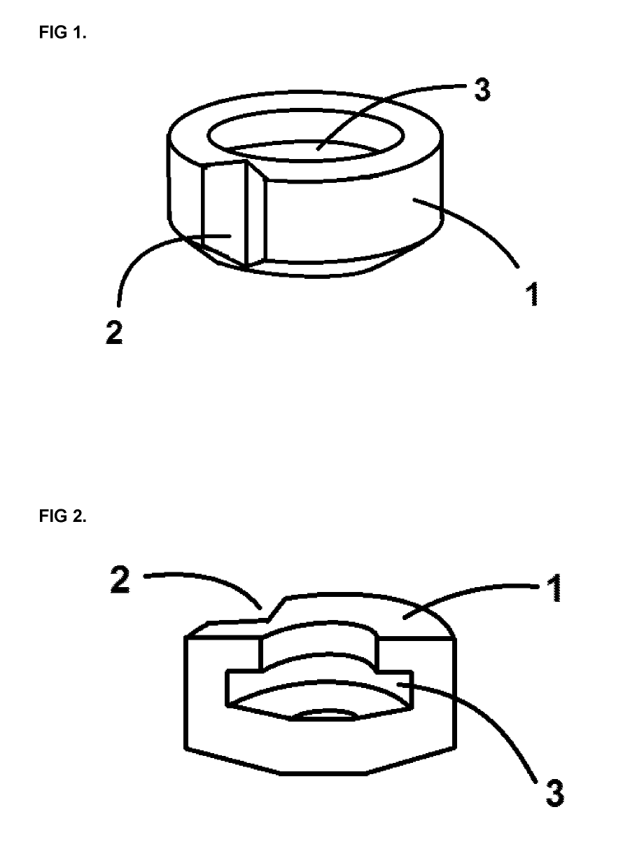

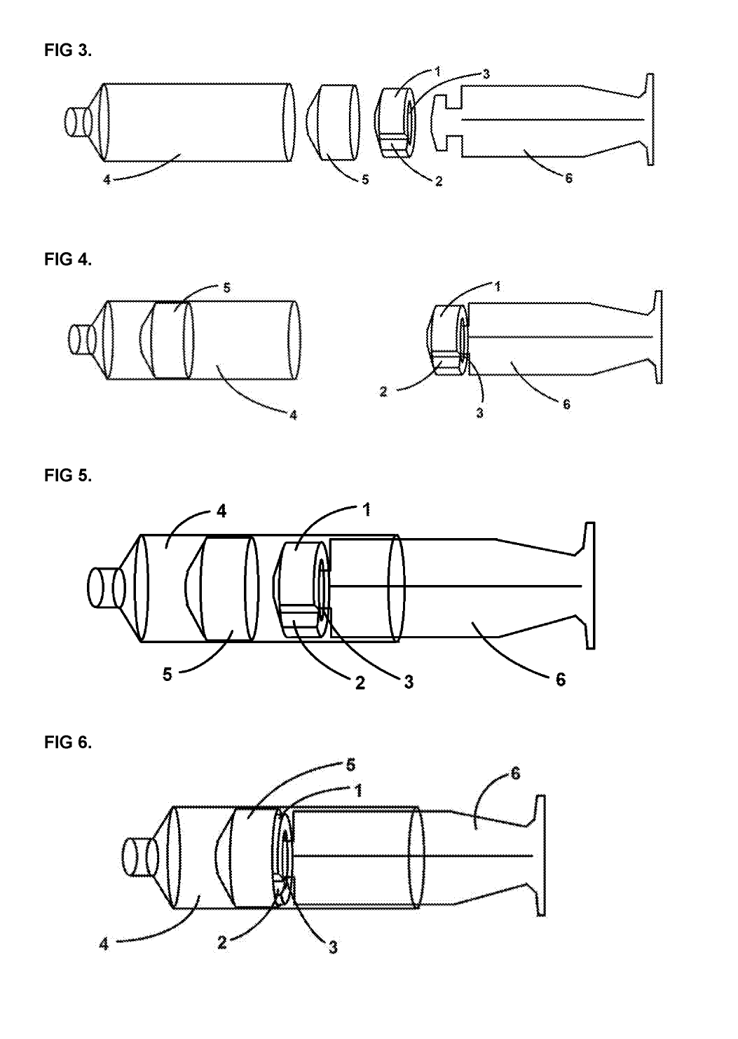

FIG. 1 shows the preferred embodiment of the nesting plunger tip 1 using a seal bypass created by the notch 2. The seal bypass can also be created by a hole or differently shaped cut-out through the nesting plunger tip 1, such that air is able to escape from between the syringe stopper 5, shown in FIG. 3, FIG. 4, FIG. 5, and FIG. 6, and the nesting plunger tip 1 when the nesting plunger tip 1 is inserted into the back end of the syringe stopper 5, which allows the plunger tip 1 to fully nest within the backside of the syringe stopper 5.

The nesting plunger tip 1 is able to fully nest within the syringe stopper 5 because the notch 2 allows air to escape as the nesting plunger tip 1 is inserted. Once the nesting plunger tip 1 is inserted, there is enough friction between the outside of the nesting plunger tip 1 and the inside of the syringe stopper 5 such that pulling back on the syringe plunger 6 causes the syringe stopper 5 to be pulled back as well, which creates a reduction in pressure on the contents of the syringe 4, which then stops the syringe 4 contents from dispensing.

FIG. 2 shows the cutaway view of the nesting plunger tip 1 which shows that the backside cavity 3 has an overhanging lip so that it holds captive the syringe plunger 6, shown in FIG. 4, once the syringe plunger 6 is inserted into the backside of the nesting plunger tip 1. The ability of the backside cavity 3 to hold the syringe plunger 6 captive is necessary to enable the syringe stopper 5 to be pulled back by pulling back on the syringe plunger 6.

To allow the syringe 4 to also be used for pneumatic dispensing, the plunger 6 can be pulled all the way out. When the plunger 6 is pulled out with sufficient force the nesting tip 1 will un-nest from within the syringe stopper 5. This then allows the back end of the syringe 4 to be attached to a pneumatic dispensing apparatus.

What is claimed is:

1. A nesting plunger tip that can nest inside a syringe stopper so that the plunger can be kept separate from the syringe so that the syringe can be used for both pneumatic dispensing or manual hand dispensing using a plunger.

2. For hand dispensing, the nesting plunger tip is pushed into place inside the back of the stopper, which then allows the plunger to be pushed into the syringe to dispense the syringe contents or pulled back from the syringe to stop dispensing the syringe contents by creating a reduced pressure inside the syringe.
